(19) United States
(12) Patent Application Publication
(10) Pub. No.: US 2022/0226554 A1
(43) Pub. Date: Jul. 21, 2022

(54) OXYGEN SUPPLYING APPARATUS HAVING CIRCULATION FACILITATING

(71) Applicant: TREATYOU MEDICAL TECHNOLOGY CORPORATION, Zhubei City (TW)

(72) Inventor: Wen-Feng Yang, Hsinchu (TW)

(21) Appl. No.: 17/341,334

(22) Filed: Jun. 7, 2021

(30) Foreign Application Priority Data
Jan. 21, 2021 (TW) 110102271

(51) Int. Cl. A61M 1/32; A61M 16/10

(52) U.S. Cl. CPC A61M 1/32; A61M 16/101; A61M 16/0672; A61M 2205/8206; A61M 2202/0208

(57) ABSTRACT

An oxygen supply apparatus is configured to directly supply oxygen-containing gas to an user for inhalation while facilitating blood circulation of a specific portion of the user. The oxygen supply apparatus includes a power supply; an oxygen-containing gas source for providing the oxygen-containing gas; a gas catheter having one end thereof connected to the oxygen-containing gas source; an inhalator connected to the other end of the gas catheter; and a circulation facilitating device at least partially attached to the specific portion of the user. The circulation facilitating device includes a sonic oscillation circuit and a housing. The sonic oscillation circuit is configured to produce oscillation at a frequency ranging from 20 Hz to 10 MHz, and the housing encloses the sonic oscillation circuit in a watertight manner

### CROSS REFERENCE TO RELATED APPLICATIONS

The present application claims priority from Taiwan Patent application No. TW 110102271 filed on Jan. 21, 2021, entitled “OXYGEN SUPPLYING APPARATUS HAVING CIRCULATION ASSISTING DEVICE”, the content of which is hereby incorporated by reference in their entirety for all purposes.

### BACKGROUND OF THE INVENTION

### Technical Field

The present invention relates to an oxygen supplying apparatus, and particularly to an oxygen supplying apparatus having an auxiliary device which facilitates local blood circulation of an user during the supplying of oxygen to the user.

### Description of the Prior Art

One primary function of an oxygen supplying apparatus is to provide oxygen-containing gas at a substantially higher oxygen concentration than that of ambient air (hereinafter, oxygen-containing gas) to an user for medical and/or healthcare purposes. Usually, the oxygen concentration in ambient air is around 20%, but in the case that the oxygen concentration is raised to about 25% to 50%, it would be helpful for a person on recovering from tiredness and pressure relief after high-intensity exercises. Moreover, oxygen-containing gas with even higher oxygen concentrations (such as 70% or more, or pure oxygen) is often used in medical or health care for respiratory diseases, for example.

Common methods for supplying oxygen-containing gas include: 1) pressure swing adsorption (PSA) methods, 2) electrolysis methods, and 3) usage of oxygen tanks. In PSA methods, oxygen is extracted from ambient air through the work of a molecular sieve mechanism in the oxygen supplying apparatus, and nitrogen in the ambient air is absorbed, so as to increase oxygen concentration. The electrolysis method utilizes liquid water electrolysis to generate oxygen and hydrogen, the generated oxygen is then extracted for use. When using oxygen tanks for supplying oxygen-containing gas, the oxygen-containing gas is stored in liquid state and under high pressure in steel cylinders used as oxygen tanks, and the stored oxygen-containing gas can be released from the tanks when it is needed.

The oxygen inhaled by the user is brought to various parts of the user's body through blood circulation. However, when the blood circulation of the user is gentle, and/or when the blood circulation at a specific body portion of the user is relatively poorer than other body portions of the user, the benefit of the oxygen supplying apparatus cannot be fully obtained due to gentle or poor blood circulation, even if the oxygen supplying apparatus provides gas having higher oxygen concentration to the user. In view of this, in order to improve the efficiency of the oxygen supplying apparatus, it is desired to facilitate blood circulation of specific body portion(s) of the user when supplying gas having higher oxygen concentration to the user.

### SUMMARY OF THE INVENTION

In one embodiment of the present invention, an oxygen supplying apparatus is provided. The oxygen supplying apparatus is configured to supply an oxygen-containing gas having an oxygen concentration equal to or higher than that of ambient air to an user for direct inhalation, and facilitate blood circulation at a specific body portion of the user in the meantime. The oxygen supplying apparatus comprises: a power supply; an oxygen-containing gas source configured to receive electrical energy from the power supply and thereby provide the oxygen-containing gas; a gas catheter having one end thereof connected to the oxygen-containing gas source; an inhalator connected to the other end of the gas catheter, the oxygen-containing gas being arranged to flow from the oxygen-containing gas source to the inhalator via the gas catheter for reaching an inhaling portion of the user; and a circulation facilitating device configured to be at least partially attached to a surface of the specific body portion of the user. The circulation facilitating device includes a sonic oscillation circuit and a housing. The sonic oscillation circuit is configured to receive electrical energy from the power supply and thereby generate oscillation at a frequency ranging from 20 Hz to 10 MHz. The housing is configured to enclose the sonic oscillation circuit in a watertight manner

In another embodiment of the present invention, the oxygen supplying apparatus further comprises a controlling section configured for controlling the oscillation of the circulation facilitating device. Further, the controlling section controls the oscillation generated by the sonic oscillation circuit of the circulation facilitating device based on predetermined voltages.

In yet another embodiment of the present invention, the oxygen supplying apparatus further comprises a potential balancing device configured for balancing a body potential of the user. The potential balancing device includes a conducting element, a grounding plate, and a lead. The conducting element is configured to be attached to the body of the user and connected to the grounding plate via the lead.

### BRIEF DESCRIPTION OF THE DRAWINGS

FIG. 1 schematically illustrates an oxygen supplying apparatus according to an embodiment of the present invention.

FIG. 2A schematically illustrates an oxygen-containing gas source according to an embodiment of the present invention.

FIG. 2B schematically illustrates a gas catheter according to an embodiment of the present invention.

FIG. 2C schematically illustrates an inhalator according to an embodiment of the present invention.

FIG. 3 schematically illustrates a circulation facilitating device according to an embodiment of the present invention.

FIG. 4 illustrates a circulation facilitating device according to another embodiment of the present invention.

FIG. 5 schematically illustrates a sonic oscillation circuit according to an embodiment of the present invention.

FIG. 6 illustrates an oxygen supplying apparatus according to another embodiment of the present invention.

In the accompanying drawings of the present invention, numeral may be repeatedly utilized to identify similar and/or same components.

### DETAILED DESCRIPTION OF THE PREFERRED EMBODIMENTS

Several preferred embodiments are set forth herein to provide better understanding of the technical features and advantages of the present invention. It should be understood that the embodiments provided in the figures are illustrative only, and may not be drawn to scale. In the figures, various details have been omitted in order not to unnecessarily obscure the present invention.

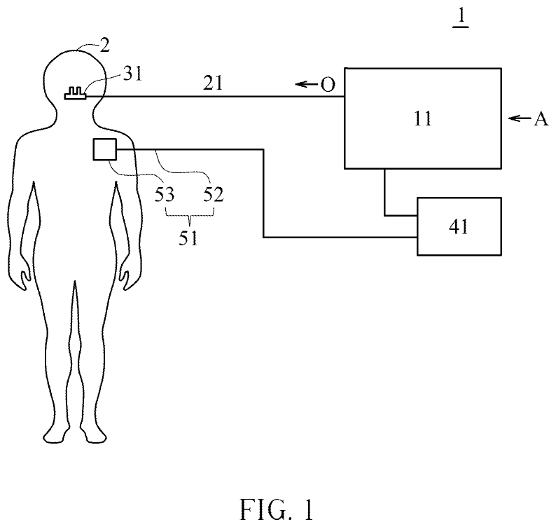

FIG. 1 illustrates an oxygen supplying apparatus 1 according to an embodiment of the present invention. As shown in FIG. 1, the oxygen supplying apparatus 1 includes an oxygen-containing gas source 11, a gas catheter 21, an inhalator 31, a power supply 41, and a circulation facilitating device 51.

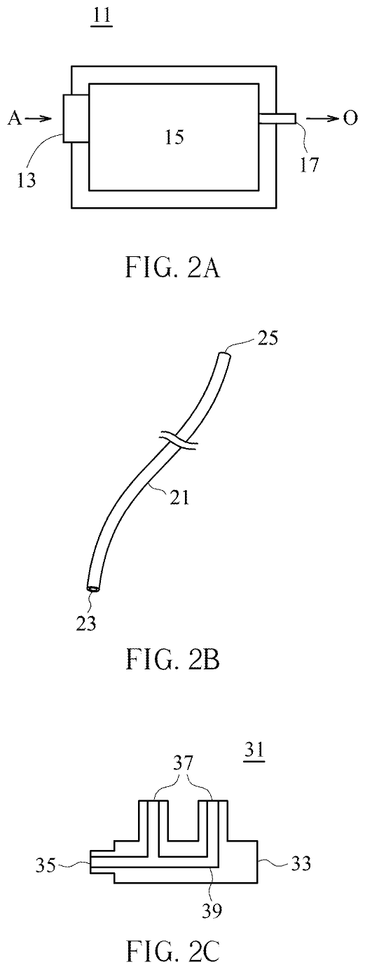

FIG. 2A illustrates the structure of the oxygen-containing gas source 11. oxygen-containing gas source 11 is configured to receive electrical energy supplied by the power supply 41, and provide oxygen-containing gas O with an oxygen concentration equal to or higher than that of ambient air. In one embodiment, the oxygen-containing gas source 11 may be a pressure swing adsorption (PSA) oxygen concentrator, which is capable of providing oxygen-containing gas O having an oxygen concentration of 33%˜42%. In another embodiment, the oxygen-containing gas source 11 may be an air compressor driven by the electricity supplied from the power supply 41.

As shown in FIG. 2A, the oxygen-containing gas source 11 includes: an inlet port 13, a molecular sieve 15, and a gas supply port 17. When the oxygen-containing gas source 11 is activated, ambient air A is sucked into the oxygen-containing gas source 11 through the inlet port 13. By using the molecular sieve 15, a portion of nitrogen is filtered out from the ambient air A sucked into the oxygen-containing gas source 11 for increasing oxygen concentration. This generates oxygen-containing gas O with an increased oxygen concentration, which will then be supplied through the gas supply port 17.

FIG. 2B illustrates the structure of the gas catheter 21. The gas catheter 21 is a hollow tube which allows the oxygen-containing gas O to flow therethrough. One end 23 of the gas catheter 21 is connected to the gas supply port 17 of the oxygen-containing gas source 11 (FIG. 2A), while the other end 25 of the gas catheter 21 is connected to a gas inlet 35 of the inhalator 31 (FIG. 2C), which will be described in detail below.

FIG. 2C illustrates a cross-sectional view of the inhalator 31. The inhalator 31 enables an user 2 to inhale the oxygen-containing gas O directly. In one embodiment, the inhalator 31 is designed to have a nose bracket shape to facilitate attachment to an inhaling portion (e.g., mouth and/or nose) of the user 2 (FIG. 1). The inhalator 31 includes an inhalator body 33, a gas inlet 35, a gas outlet 37, and an internal duct 39. As shown in FIG. 2C, the inhalator body 33 is provided with the gas inlet 35, at least one gas outlet 37 and the internal duct 39. As mentioned above, the gas inlet 35 is configured to be connected to one end 25 of the gas catheter 21, allowing the oxygen-containing gas O to flow into the inhalator 31. The gas outlet 37 may be connected to the inhaling portion (e.g., mouth and/or nose) of the user 2, allowing the user 2 to inhale the oxygen-containing gas O directly. In an embodiment, the gas outlet 37 may be positioned in nostril(s) of the user 2. The internal duct 39 is formed inside the inhalator body 33, and connected to the gas inlet 35 and gas outlet 37. For example, the internal duct 39 may be formed as a hollow portion of the inhalator body 33.

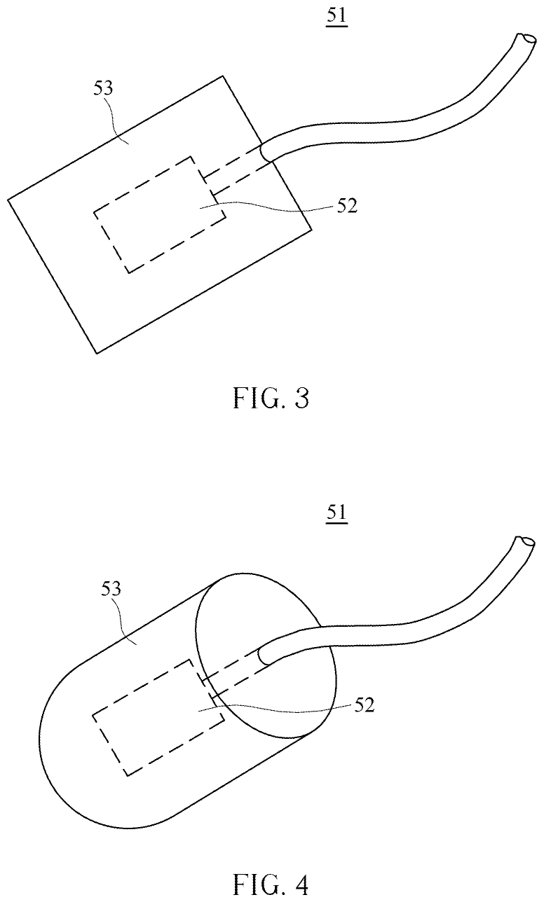

FIG. 3 illustrates the structure of the circulation facilitating device 51. As shown in FIG. 3, the circulation facilitating device 51 includes a sonic oscillation circuit 52 and a housing 53. One end of the circulation facilitating device 51 is connected to the power supply 41, so the sonic oscillation circuit 52 of the circulation facilitating device 51 can generate sonic oscillation using the electrical energy provided by the power supply 41, as will be described in detail below. The housing 53 encloses the sonic oscillation circuit 52, and has an opening that allows the end of the sonic oscillation circuit 52 connected to the power supply 41 to penetrate therethrough. The housing 53 encloses the sonic oscillation circuit 52 in a water-tight manner, in order to keep moisture from entering the inside of the housing 53 and prevent the sonic oscillation circuit 52 from being damaged by moisture. The housing 53 may be made of, such as, but not limited to silicone rubber. Although in FIG. 3, the housing 53 is shown to be rectangular in its shape, it can be formed into various shapes, depending on different specific body portions of the user to which the circulation facilitating device 51 is to be applied. The housing may be formed to be, for example, but not limited to cylindrical (as shown in FIG. 4), hemispherical, etc., in its shape. Although it is shown in FIG. 3 and FIG. 4 that one sonic oscillation circuit 52 is provided within the housing 53, this is only illustrative, and there may be a plurality of sonic oscillation circuits 52 provided within the housing 53. Further, as an example, the circulation facilitating device 51 is attached to the oxygen supplying apparatus 1 in a detachable manner, but it is not limited thereto.

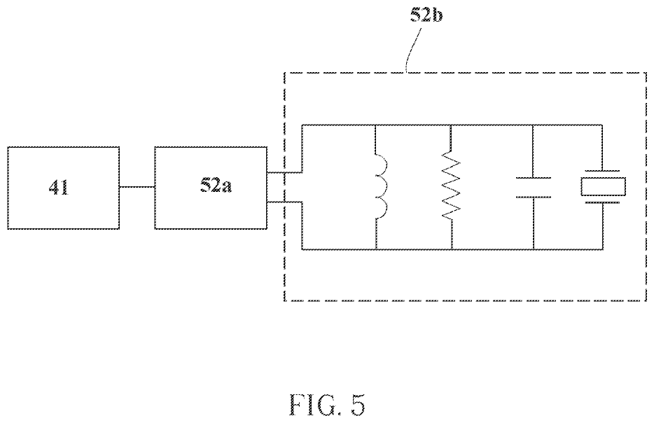

FIG. 5 illustrates an exemplary construction for the sonic oscillation circuit 52 of the circulation facilitating device 51. In the embodiment of FIG. 5, the sonic oscillation circuit 52 is a digital sonic oscillation circuit, but this is only illustrative, and other sonic oscillation circuits may be used. As shown in FIG. 5, the sonic oscillation circuit 52 includes a controlling section 52a and a piezoelectric oscillating element 52b, where the piezoelectric oscillating element 52b is coupled to the power supply 41 through the controlling section 52a. The controlling section 52a may be a digital oscillation controlling circuit, but it is not limited thereto. The piezoelectric oscillating element 52b produces piezoelectric effect using the electric field generated by the power supply 41, thereby causing electrostriction. In this manner, when predetermined frequencies and/or voltages are regularly applied to the piezoelectric oscillation element 52b, the piezoelectric oscillation element 52b can be caused to produce mechanical vibrations (i.e., oscillations). Moreover, in order to make the oscillation of the piezoelectric oscillation element 52b meet the requirements of the user 2, the controlling section 52a coupled between the power supply 41 and the piezoelectric oscillation element 52b can receive an electric field from the power supply 41 for generating predetermined voltages, and output these predetermined voltages to the piezoelectric oscillation element 52b. This causes the piezoelectric oscillation element 52b to oscillate in a resonant manner, and predetermined oscillation frequencies and intensities can thus be provided to the user 2. Since the piezoelectric oscillation element 52b is employed in the sonic oscillation circuit 52 as an element for producing oscillations having desired frequencies and intensities, the sonic oscillation circuit 52 of the present invention can be quiet, downsized and lightweight, in comparison with conventional oscillation devices provided with vibration motors.

The oscillation frequency of human bodies falls within a range from 1 Hz to less than 20 Hz, so it is desired that the sonic oscillation circuit 52 produces frequencies higher than the oscillation frequency of human bodies. For example, the oscillation frequency produced by the sonic oscillation circuit 52 can be in the range of 20 Hz˜10 MHz, preferably 20 Hz˜2 MHz, and more preferably 20 Hz˜20 KHz.

An operational process of the oxygen supplying apparatus 1 is explained below.

First, the inhalator 31 is attached to a corresponding body portion of the user 2, so that the gas outlet is connected to the user 2. Next, the user 2 can place the circulation facilitating device 51 to a specific body portion where circulation facilitation is required, such as but not limited to, arms, shoulders, neck, back, legs, mouth, anus, vagina, etc. When being placed to the specific body portion, the circulation facilitating device 51 is at least partially attached to the surface of the specific body portion. During the operation of the oxygen supplying apparatus 1, the oxygen-containing gas source 11 generates the oxygen-containing gas O as described above, and provides the oxygen-containing gas O through the gas supply port 17. The oxygen-containing gas O is then flowed to the inhalator 31 through the gas catheter 21, allowing the user 2 to inhale the oxygen-containing gas O directly. Also, during the operation of the oxygen supplying apparatus 1, the circulation facilitating device 51 receives electrical energy from the power supply 41 for causing the sonic oscillation circuit 52 to produce sonic oscillations, and transfer such sonic oscillations to the specific body portion of the user 2 where the circulation facilitating device 51 is attached to. Thus, the blood circulation at the specific body portion can be improved. In this manner, oxygen in the inhaled oxygen-containing gas O can arrive the specific body portion sooner, and the overall performance of the oxygen supply apparatus 1 can therefore be effectively improved.

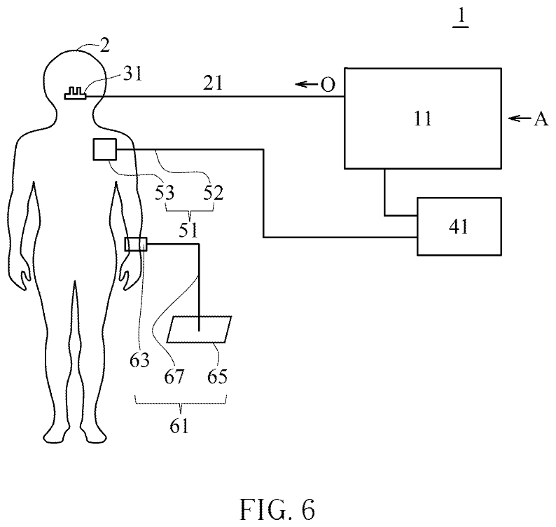

FIG. 6 illustrates an oxygen supply apparatus according to another embodiment of the present invention. In the embodiment shown in FIG. 6, the oxygen supplying apparatus 1 further includes a potential balancing device 61. The potential balancing device 61 includes: a conducting element 63, a grounding plate 65, and a lead 67. In the oxygen supplying apparatus 1 shown in FIG. 6, the conducting element 63 is formed of a conductor having good electrical conductivity, and connected to the grounding plate 65 via the lead 67. Specifically, the conducting element 63 may be formed of metal, for example, and is preferably formed of materials which will not result in any allergic reaction of the user 2, such as gold, silver, platinum, titanium, alloys thereof, etc. With such a configuration, during the operation of the oxygen supplying apparatus, the conducting element 63 can be placed, for example, directly on the skin of the user 2. The grounding plate 65 is formed of a conductor having good electrical conductivity and a large volume, so that the balancing for a body potential V of the user 2 can be performed using the potential balancing effect of such a large volume conductor. The grounding plate 65 may additionally be connected to ground outside the potential balancing device 61. From the aspect of safety, it is preferable to provide safety protection measures by employing Zener diodes or the like in the potential balancing device 61. For example, the potential balancing device 61 may be attached to the oxygen supplying apparatus 1, or may be a built-in device of the oxygen supplying apparatus 1.

An operational process of the potential balancing device 61 is described below. When the conducting element 63 is placed on, for example, the skin of the user 2, the body potential V of the user 2 can be conducted from the conducting element 63 to the grounding plate 65 via the lead 67, and the body potential of the user 2 can thereby be balanced.

With the aforementioned configuration, it is possible for the oxygen supplying apparatus 1 to keep the body potential of the user 2 balanced, while supplying oxygen-containing gas having an oxygen concentration equal to or higher than that of ambient air to the user 2. Thus, the blood circulation of the user 2 can be facilitated, and the benefit of healthcare of the oxygen supplying apparatus 1 can also be maximized

In one embodiment, the oxygen supplying apparatus 1 is designed to be portable, and the power supply 41 for such an oxygen supplying apparatus 1 may be a battery. With this configuration, the user will be able to store the oxygen supplying apparatus 1 in a carrying bag or a carrying box for carrying the oxygen supplying apparatus 1 around, and take the oxygen supplying apparatus 1 out of the carrying bag or carrying box when required, without being limited by factors such as places or power sources.

Although the present invention has been described in detail with reference to the preferred embodiments and drawings, those with ordinary skill in the art may understand that various modifications, changes, and equivalents can be made without departing from the spirit and scope of the present invention. However, these modifications, changes, and equivalents should also be included in the scope of the present invention.

1. An oxygen supplying apparatus configured for supplying an oxygen-containing gas having an oxygen concentration equal to or higher than that of ambient air to an user and facilitating blood circulation at a specific body portion of the user, the oxygen supplying apparatus comprising:
a power supply;
an oxygen-containing gas source configured to receive electrical energy from the power supply and thereby provide the oxygen-containing gas;
a gas catheter having one end thereof connected to the oxygen-containing gas source;
an inhalator connected to the other end of the gas catheter, the oxygen-containing gas being arranged to flow from the oxygen-containing gas source to the inhalator via the gas catheter for reaching an inhaling portion of the user; and
a circulation facilitating device configured to be at least partially attached to a surface of the specific body portion of the user, the circulation facilitating device comprising a sonic oscillation circuit and a housing, wherein the sonic oscillation circuit is configured to receive electrical energy from the power supply and thereby generate oscillation at a frequency ranging from 20 Hz to 10 MHz, and the housing is configured to enclose the sonic oscillation circuit in a watertight manner

2. The oxygen supplying apparatus of claim 1, wherein the oxygen-containing gas source is a pressure swing adsorption (PSA) oxygen concentrator.

3. The oxygen supplying apparatus of claim 1, wherein the sonic oscillation circuit comprises a piezoelectric oscillating element.

4. The oxygen supplying apparatus of claim 1, wherein the oscillation generated by the sonic oscillation circuit is at a frequency ranging from 20 Hz to 2 MHz.

5. The oxygen supplying apparatus of claim 4, wherein the oscillation generated by the sonic oscillation circuit is at a frequency ranging from 20 Hz to 20 KHz.

6. The oxygen supplying apparatus of claim 1, wherein the housing is made of silicone rubber.

7. The oxygen supplying apparatus of claim 1, wherein the circulation facilitating device is attached to the oxygen supplying apparatus in a detachable manner

8. The oxygen supplying apparatus of claim 1, further comprising:
a controlling section configured for controlling the oscillation of the circulation facilitating device, wherein the controlling section controls the oscillation generated by the sonic oscillation circuit of the circulation facilitating device based on predetermined voltages.

9. The oxygen supplying apparatus of claim 1, further comprising:
a potential balancing device configured for balancing a body potential of the user and comprising a conducting element, a grounding plate, and a lead, wherein the conducting element is configured to be attached to the body of the user and connected to the grounding plate via the lead.
